(19) United States
(12) Patent Application Publication
(10) Pub. No.: US 2008/0113973 A1
(43) Pub. Date: May 15, 2008

(54) Compositions and methods involving the combination of a thromboxane A2 receptor antagonist and an inhibitor of cyclooxygenase-2

(75) Inventor: Hans R. Brunner, Pully VD (CH)

Correspondence Address: LAW OFFICE OF MICHAEL A. SANZO, LLC, 15400 CALHOUN DR., SUITE 125, ROCKVILLE, MD, 20855, US

(73) Assignee: B.M.R.A. Corporation B.V., Rotterdam (NL)

(21) Appl. No.: 11/979,450

(22) Filed: Nov. 2, 2007

Related U.S. Application Data
(62) Division of application No. 10/612,338, filed on Jul. 3, 2003, now abandoned.
(60) Provisional application No. 60/394,268, filed on Jul. 9, 2002.

(51) Int. Cl. A61K 31/5415; A61K 31/557; A61K 31/415; A61K 31/365

(52) U.S. Cl. 514/222.8; 514/406; 514/471; 514/573

(57) ABSTRACT

The invention is directed to methods and compositions that can be used in the treatment of inflammation, pain and cardiovascular disorders. Methods and compositions are described involving the combination of a thromboxane A2 receptor antagonist and an inhibitor specific for cyclooxygenase-2.

### CROSS REFERENCE TO RELATED APPLICATIONS

This application claims priority to U.S. provisional application No. 60/394,268, filed on Jul. 9, 2002, which is incorporated in its entirety herein by reference.

### FIELD OF THE INVENTION

The invention is directed to compositions containing both a cyclooxygenase-2 (COX-2) inhibitor and a thromboxane A2 receptor antagonist. The compositions may be used to treat patients for pain or inflammation and have less risk of inducing adverse cardiovascular effects than when COX-2 inhibitors are administered alone. The invention includes not only these compositions, but also methods in which patients are treated.

### BACKGROUND OF THE INVENTION

COX-2 Specific Inhibitors

Over 15 million Americans take nonsteroidal anti-inflammatory drugs (NSAIDs) each day as a treatment for pain or inflammation. Unfortunately, many of these drugs are also associated with a high incidence of gastrointestinal complications, including gastritis, dyspepsia, gastroduodenal ulcers, perforations, and bleeding. As a result, it has been estimated that as many as 15,000 people in the U.S. die each year from taking NSAIDs (www.emedmag.com/stories/storyReader$118).

Most NSAIDS exert their effects by nonselectively blocking two enzymes, cyclooxygenase-1 (COX-1) and cyclooxygenase-2 (COX-2). It appears that inhibition of COX-2 is primarily responsible for alleviating pain and inflammation, whereas inhibition of COX-1 is primarily responsible for damage to the GI tract (Vane, et al., Am. J. Med. 104:2 S-8S (1998)). As a result, inhibitors specific for COX-2 have been developed and some are now on the market. These drugs maintain the ability to alleviate pain but are safer with respect to adverse gastrointestinal effects (Griswold, et al., Med. Res. Rev. 16(2): 181-206 (1996); Lane, J. Rheumatol 24 (Suppl 49):20-4 (1997); Lipsky, et al., J. Rheumatol. 24(Suppl 49):9-14 (1997)).

More recent research has led many to reconsider the wisdom of blocking one cyclooxygenase enzyme but not the other (Mukherjee, et al., JAMA 286:954-959 (2001); Science 296:539-541 (2002)). COX-1, makes thromboxane, which causes blood vessels to constrict and platelets to become sticky. These activities can contribute to a heart attack or stroke. In contrast, COX-2 promotes the production of prostacyclin which dilates blood vessels and prevents platelets from clumping together. In a normal person, the two enzymes appear to balance one another. COX-2 specific inhibitors upset this balance by only blocking the production of prostacyclin while allowing thromboxane production to remain unchecked. As a result, the COX-2 inhibitors increase the risk of adverse cardiovascular events.

Thromboxane A2 Receptor Antagonists

Thromboxane A2/prostaglandin H2 receptor antagonists have been reported to be effective in treating, inter alia, arterial or venous thrombosis, unstable angina, transient ischemic attacks, and hypertension, (U.S. Pat. No. 5,100,889). They include 7-oxabicycloheptane substituted prostaglandin analogs (U.S. Pat. No. 5,100,889; Rosenfeld, et al., Cardiovascular Drug Rev. 19:97-115 (2001)), benzenealkonic acids (U.S. Pat. No. 5,618,941), and benzenesulfonamide derivatives (U.S. Pat. No. 5,597,848). In general, these compounds have not been reported to directly affect either cyclooxygenase-1 or cyclooxygenase-2.

### SUMMARY OF THE INVENTION

The present invention is based upon the concept that the cardiovascular risks associated with the administration of COX-2 specific inhibitors can be avoided by co-administering an agent that blocks the activation of the thromboxane A2 receptor by its ligand. The invention includes compositions, therapeutic packages and treatment methods.

In its first aspect, the invention is directed to a pharmaceutical composition in unit dose form which contains a COX-2 inhibitor and a thromboxane A2 receptor antagonist. Both of these drugs are present in an amount that is therapeutically effective upon the administration of one or more unit doses of the composition to a patient. The term “unit dose” or “unit dose form” refers to a single drug administration entity. By way of example, a single tablet, capsule, dragee, vial for injection or syringe combining both a COX-2 inhibitor and a thromboxane A2 receptor antagonist would be a unit dose form. As used herein, the term “COX-2 inhibitor” refers to agents that specifically inhibit COX-2 and which have little or no effect on COX-1. For example, at a dosage that caused a 50% inhibition of COX-2, a COX-2 inhibitor would inhibit COX-1 by less than 10%. The term “therapeutically effective” means that sufficient drug is present to generate the therapeutic action for which the drug is given. For example, if a patient is being treated for pain then a “therapeutically effective” amount of COX-2 would be a dosage sufficient to reduce the severity or duration of the pain. If the patient is being treated for inflammation, then enough drug would need to be present to reduce the associated pain or swelling. In the case of thromboxane A2 receptor inhibitors, enough should be present to treat or prevent cardiovascular problems associated with thromboxane A2. This means that, in general between 0.1 mg and 500 mg., (and preferably between 1 and 100 mg) will be present.

Preferred COX-2 inhibitors for use in the compositions are celecoxib; rofecoxib; meloxicam; JTE-522; L-745,337; NS398. Thromboxane A2 receptor antagonists include 7-oxabicycloheptane substituted prostaglandin analogs such as those described in U.S. Pat. No. 5,100,889, benzenealkonic acids and benzenesulfonamide derivatives. The most preferred drugs are ifetroban and either celecoxib or rofecoxib. It will be understood that, unless otherwise indicated, reference to a COX-2 inhibitor or thromboxane A2 receptor antagonist includes all pharmaceutically acceptable forms of the drug known in the art. For example, any pharmaceutically acceptable salt of a drug may be used in compositions. In general, the COX-2 inhibitor will be present at between 1 and 500 mg.

The therapeutic agents described above, i.e., the COX-2 inhibitor and the thromboxane A2 receptor antagonist, may be supplied in the form of a therapeutic package. Each package has one or more finished pharmaceutical containers with the therapeutic agents in unit dose form and includes labeling directed to their use in the treatment of any condition responsive to a COX-2 inhibitor or a thromboxane A2 receptor antagonist, These conditions include inflammation (e.g., that associated with arthritis); pain (e.g., pain associated with headache, muscle pain, or post-surgical pain); and cardiovascular conditions (e.g., arterial or venous thrombosis, angina, or hypertension).

The invention also includes methods of treating a patient for any condition responsive to a COX-2 inhibitor or a thromboxane A2 receptor antagonist by either administering the pharmaceutical compositions described above or by sequentially administering the two drugs in a co-timely manner, i.e., the second drug is administered while the first drug is still present in a therapeutically effective amount. Any of the specific conditions mentioned above may be treated in this manner. The preferred agents are ifetroban and either celecoxib or rofecoxib.

### DETAILED DESCRIPTION OF THE INVENTION

A. COX-2 Inhibitors and Thromboxane A2 Receptor Antagonists

The GI toxicity associated with many NSAIDs appears to be due to the inhibition COX-1 whereas anti-inflammatory effects are due to primarily to inhibition of COX-2. Drugs which selectively inhibit the COX-2 isozyme, e.g., celecoxib, rofecoxib, meloxicam, piroxicam, JTE-522 and L-745,337, produce analgesia and reduce inflammation without damaging the gastrointestinal tract.

Although, as discussed above, COX-2 specific inhibitors reduce the risk of gastrointestinal complications relative to NSAIDs inhibiting both COX-1 and COX-2, they increase the risk of serious cardiovascular problems due to the continued generation of thromboxane in the absence of normal levels of prostacyclin. The present invention addresses this problem by including a thromboxane A2 receptor antagonist in therapeutic compositions and methods.

COX-2 inhibitors have been thoroughly described in the art and some (e.g., celecoxib and rofecoxib) are now commercially available as therapies. Similarly, a variety of thromboxane A2 receptor antagonists have been disclosed and methods for synthesizing these compounds have been described for bicycloheptane substituted prostaglandin analogs (U.S. Pat. No. 5,100,889; Rosenfeld, et al., Cardiovascular Drug Rev. 97-115 (2001)), benzenealkonic acids (U.S. Pat. No. 5,618,941), and benzenesulfonamide derivatives (U.S. Pat. No. 5,597,848). Any of these prior methods may be used to obtain agents suitable for use in the present invention.

B. Route of Administration

The methods and compositions discussed above are compatible with any dosage form or route of administration. Thus, agents may be administered orally, intranasally, rectally, sublingually, buccally, parenterally, or transdermally. Dosage forms may include tablets, trochees, capsules, caplets, dragees, lozenges, parenterals, liquids, powders, and formulations designed for implantation or administration to the surface of the skin. In general, it is expected that oral dosage forms will be the most convenient. All dosage forms may be prepared using methods that are standard in the art (see e.g., Remington's Pharmaceutical Sciences, 16th ed. A. Oslo. ed., Easton, Pa. (1980)).

Active ingredients may be used in conjunction with any of the vehicles and excipients commonly employed in pharmaceutical compositions, e.g., talc, gum arabic, lactose, starch, magnesium stearate, cocoa butter, aqueous or non-aqueous solvents, oils, paraffin derivatives, glycols, etc. Coloring and flavoring agents may also be added to preparations designed for oral administration. Solutions can be prepared using water or physiologically compatible organic solvents such as ethanol, 1-2 propylene glycol, polyglycols, dimethyl sulfoxide, fatty alcohols, triglycerides, partial esters of glycerin, and the like. Parenteral compositions containing active ingredients may be prepared using conventional techniques and include sterile isotonic saline, water, 1,3-butanediol, ethanol, 1,2-propylene glycol, polyglycols mixed with water, Ringer's solution, etc.

The COX-2 inhibitors are especially useful in the treatment of pain, e.g., pain due to migraine headache, and inflammation. Thus, the invention includes methods of treating these conditions by administering a thromboxane A2 receptor antagonist in combination with a COX-2 inhibitor. These agents should be given in a co-timely manner and should be delivered in an amount sufficient to reduce pain or inflammation. In general, it is expected that the drugs will be given within 24 hours of one another.

C. Dosages

With respect to therapeutic agents, it is expected that the skilled practitioner will adjust dosages on a case by case basis using methods well established in clinical medicine. Nevertheless, the following general guidelines with respect to two preferred COX-2 inhibitors and the most preferred thromboxane A2 receptor antagonist may be of help.

Celecoxib (Celebrex®) is particularly useful when contained in tablets of from about 100 to 200 mg. Recommended dosages are typically 100 mg twice per day or 200 mg once per day (see, Bolten, J., Rheumatolog. Suppl., 51:2-7 (May, 1998)). Celecoxib is a preferred COX-2 inhibitor in the compositions and methods of the present invention and should typically be present at 50-500 mg per unit dose. Especially preferred are methods and compositions utilizing 10 to 100 mg of ifetroban and 100 to 400 mg celecoxib.

Rofecoxib (Vioxx®) for oral administration is available in tablets of 12.5, 25 or 50 mg and in an oral suspension containing either 12.5 mg or 25 mg rofecoxib per 5 ml. The recommended initial daily dosage for the management of acute pain is 50 mg. Peak plasma concentrations of rofecoxib typically occur about 2-3 hours after oral administration and the drug has a half life of about 17 hours.

The thromboxane A2 receptor antagonist should be present at a level sufficient to treat cardiovascular disease as suggested in the various patent publications cited above. In the case of ifetroban, between 1 mg/kg/day and 100 mg/kg/day should typically be given. If desired, the agents may also be given to treat any of the cardiovascular problems that have been disclosed as being amenable to treatment with thromboxane A2 receptor antagonists.

The daily dosage may be provided in either a single or multiple regimen with the latter being generally preferred. These are simply guidelines since the actual dose must be carefully selected and titrated by the attending physician based upon clinical factors unique to each patient. The optimal daily dose will be determined by methods known in the art and will be influenced by factors such as the age of the patient, the disease state, side effects associated with the particular agent being administered and other clinically relevant factors. In some cases, a patient may already be taking medications at the time that treatment with the present combination is initiated. These other medications may be continued provided that no unacceptable adverse side effects are reported by the patient.

All references cited herein are fully incorporated by reference. Having now fully described the invention, it will be understood by one of skill in the art that the invention may be performed within a wide and equivalent range of conditions, parameters and the like, without affecting the spirit or scope of the invention or any embodiment thereof.

1-29. (canceled)

30. In method of treating a patient for pain or inflammation by administering to said patient a cyclooxygenase-2 (COX-2) inhibitor, the improvement comprising cotimely administering to said patient, a therapeutically effective amount of a thromboxane A2 receptor antagonist.

31. The improvement of claim 30, wherein said patient is treated for pain associated with headache.

32. The improvement of claim 30, wherein said patient is treated for muscle pain.

33. The improvement of claim 30, wherein said patient is treated for post-surgical pain.

34. The improvement of claim 30, wherein said patient is treated for pain or inflammation associated with arthritis.

35. The improvement of claim 30, wherein said COX-2 inhibitor and said thromboxane A2 receptor antagonist are administered together in unit dose form.

36. The improvement of claim 35, wherein said unit dosage form is a tablet or capsule.

37. The improvement of claim 36, wherein said thromboxane A2 receptor antagonist is ifetroban and wherein said COX-2 inhibitor and said ifetroban are each present in said unit dose form in an amount of between 5 and 500 mg.

38. The improvement of claim 37, wherein said COX-2 inhibitor is celecoxib.

39. The improvement of claim 37, wherein said COX-2 inhibitor is rofecoxib.

40. A method for reducing the cardiovascular risk caused by a patient taking a COX-2 inhibitor for pain or inflammation, comprising concurrently administering to said patient a thromboxane A2 receptor antagonist.

41. The method of claim 40, wherein said patient is taking said COX-2 inhibitor as a treatment for pain associated with headache.

42. The method of claim 40, wherein said patient is taking said COX-2 inhibitor as a treatment for muscle pain.

43. The method of claim 40, wherein said patient is taking said COX-2 inhibitor as a treatment for post-surgical pain.

44. The method of claim 40, wherein said patient is taking said COX-2 inhibitor as a treatment for pain or inflammation associated with arthritis.

45. The method of claim 40, wherein said COX-2 inhibitor and said thromboxane A2 receptor antagonist are administered together in unit dose form.

46. The method of claim 45, wherein said unit dosage form is a tablet or capsule.

47. The method of claim 46, wherein said thromboxane A2 receptor antagonist is ifetroban and wherein said COX-2 inhibitor and said ifetroban are each present in said unit dose form in an amount of between 5 and 500 mg.

48. The method of claim 47, wherein said COX-2 inhibitor is celecoxib.

49. The method of claim 47, wherein said COX-2 inhibitor is rofecoxib.
